(19) United States
(12) Patent Application Publication
(10) Pub. No.: US 2026/0286343 A1
(43) Pub. Date: Sep. 24, 2026

(54) METHOD OF CONSTRUCTING A SPATIALLY BARCODED SURFACE

(71) Applicant: Cellanome, Inc., Foster City, CA (US)

(72) Inventors: Anirudh VENUGOPAL, Morgan Hill, CA (US); Paul KITABJIAN, San Diego, CA (US); Ali AGAH, Redwood City, CA (US)

(21) Appl. No.: 19/684,874

(22) Filed: May 21, 2026

Related U.S. Application Data
(63) Continuation of application No. PCT/US24/56910, filed on Nov. 21, 2024.
(60) Provisional application No. 63/602,180, filed on Nov. 22, 2023.

(51) Int. Cl. C12N 15/10; C40B 40/06

(52) U.S. Cl. CPC C12N 15/1065; C40B 40/06

(57) ABSTRACT

A method of making a barcoded surface includes synthesizing a plurality of arrays arranged in rows and columns. Each array can include a plurality of discrete reaction sites with a first oligonucleotide containing a first unique array barcode sequence. The first unique array barcode sequences of the first oligonucleotides corresponds to a mapped discrete reaction site location within the array. A second oligonucleotide can be coupled to the first oligonucleotide that contains a second unique barcode sequence. The first unique barcode sequences of the second oligonucleotides corresponds to a mapped first sub-group location within the plurality of arrays.

### CROSS-REFERENCE TO RELATED APPLICATIONS

This application is a continuation of International Application No. PCT/US2024/056910, filed Nov. 21, 2024, which claims the benefit of U.S. Provisional Application No. 63/602,180, filed Nov. 22, 2023, each of which are incorporated herein by reference in its entirety.

### BACKGROUND

Molecular tagging has a long history in analytical biochemistry and molecular biology, e.g. Church U.S. Pat. No. 4,942,124; Spitzer et al, Cell, 165(4): 780-791 (2016); Giese, Trends in Analytical Chemistry, 2(7): 166-168 (1983); Hardenbol et al, Nature Biotechnology, 21: 673-678 (2003); Brenner et al, U.S. Pat. No. 7,537,897; Fan et al, Science, 347 (6222): 1258367-1 (2015); Macevicz, U.S. patent publication US2005/0250147; Morris et al, European patent publication 0799897A1; Wallace, U.S. Pat. No. 5,981,179; and the like. Recently, such techniques have been expanded to include the use of spatially distributed oligonucleotide barcodes for identifying and studying spatial variations in biological processes, such as tissue-wide gene expression, e.g. Stahl et al, Science, 353(6294): 78-82 (2016); Salmen et al, Nature Protocols, 13: 2501-2534 (2018); Frisen et al, U.S. Pat. No. 9,593,365; and the like. However, cost effective synthesis of spatial barcodes with known sequences, control of spatial barcode distributions, and densities for resolving cellular and subcellular processes have been a challenge. This challenge has been addressed with only partial success by a plethora of different approaches, e.g. Horgan et al, International patent publication, WO/2022/013094; Liu et al, Cell, 183: 1665-1681 (2020); Cho et al, bioRxiv (https://doi.org/10.1101/2021.01.25.427004); Chen et al (https://doi.org/10.1101/2021.01.17.427807); Delly et al, Scientific Reports, 11: 10857(2021 ); Rodriques et al, Science, 363(6434): 1463-1467 (2019); and the like. The field of spatial barcode construction would be advanced by the availability of a cost effective spatial barcoding method.

### SUMMARY

The systems, devices, and methods described herein are directed to making spatially barcoded surfaces and nucleic acid molecules using combinatorial techniques. A method of making a barcoded surface includes synthesizing a plurality of arrays. Each array includes a plurality of discrete reaction sites. Each discrete reaction site includes a first oligonucleotide coupled to a surface of a solid support at a 5′ end of the first oligonucleotide. The surface includes an uncoated region in between neighboring arrays. The uncoated region includes an area where no first oligonucleotides are coupled to the surface. The first oligonucleotide includes a first unique array barcode sequence for each discrete reaction site location within the array. Each of the first unique array barcode sequences of the first oligonucleotides corresponds to a mapped discrete reaction site location within the array. A liquid containing a second oligonucleotide is disposed onto each array of the plurality of arrays where a portion of the uncoated region in between neighboring arrays is not covered by the liquid containing the second oligonucleotide. The second oligonucleotide couples to the first oligonucleotide to form a first surface oligonucleotide complex. The second oligonucleotide includes a second unique barcode sequence. The second unique barcode sequence includes a same sequence within a first sub-group of the plurality of arrays and a different sequence between each of the first sub-groups of the plurality of arrays. Each of the second unique barcode sequences of the second oligonucleotides correspond to a mapped first sub-group location within the plurality of arrays.

In regard to various methods, the methods can further include disposing another liquid containing a third oligonucleotide onto each array of the plurality of arrays where a portion of the uncoated region in between neighboring arrays is not covered by the another liquid containing the third oligonucleotide. The third oligonucleotide couples to the second oligonucleotide to form a second surface oligonucleotide complex. The third oligonucleotide includes a third unique barcode sequence. The third unique barcode sequence includes a same sequence within a second sub-group of the plurality of arrays and a different sequence between each of the second sub-groups of the plurality of arrays. Each of the third unique barcode sequences of the third oligonucleotides corresponds to a mapped second sub-group location within the plurality of arrays.

In regard to various methods, the second oligonucleotide can further include a random barcode sequence that is a unique sequence for at least 99% of the formed second surface oligonucleotide complexes.

In regard to various methods, the disposing the liquid containing the second oligonucleotide onto each array of the plurality of arrays can include disposing a puddle of the liquid onto each array, wherein each puddle does not touch neighboring puddles.

In regard to various methods, each puddle can be a single body of the liquid.

In regard to various methods, the puddle is formed by disposing the liquid as a plurality of dispensed droplets that merge together.

In regard to various methods, the plurality of dispensed droplets have a pitch along the x axis ranging from about 1 micron to about 40 microns, and a pitch along the y axis ranging from about 1 micron to about 40 microns.

In regard to various methods, the droplets are formed from an ink jet nozzle or piezoelectric pump.

In regard to various methods, each of the droplets can have a diameter ranging from about 80 microns to about 140 microns.

In regard to various methods, the each of the droplets can have a volume ranging from about 200 picoliters to about 350 picoliters.

In regard to various methods, the disposing the liquid containing the second oligonucleotide onto each array of the plurality of arrays can include disposing the liquid as a plurality of droplets, wherein each droplet at least partly covers each of the discrete reaction sites. In various methods, each droplet is a single droplet that at least partly covers each of the discrete reaction sites.

In regard to various methods, the disposing the liquid containing the second oligonucleotide onto each array of the plurality of arrays can include disposing the liquid as a plurality of droplets, wherein each droplet covers each of the discrete reaction sites. In various methods, each droplet is a single droplet that covers each of the discrete reaction sites.

In regard to various methods, the synthesizing a plurality of arrays can include coupling an amine moiety of the first oligonucleotide with the surface via a coupling layer, wherein an amine reactive moiety of the coupling layer binds to the amine moiety.

In regard to various methods, the surface can include a glass surface and the coupling layer can include a silane linkage to the glass surface and the amine reactive moiety can include N-hydroxysuccinimide moiety.

In regard to various methods further including the after coupling the second oligonucleotide to the first oligonucleotide to form the first surface oligonucleotide complex, adding a ligase to extend the first oligonucleotide so that a portion of the second oligonucleotide is copied.

In regard to various methods, the plurality of arrays is arranged in rows and columns.

In regard to various methods, the rows and the columns of the plurality of arrays are arranged in an orthogonal relationship.

In regard to various methods, each of the discrete reaction sites of the array are formed from a single disposed droplet containing the first oligonucleotides.

In regard to various methods, each of the arrays of the plurality can have a same patterned location of the first unique array barcode sequences of the first oligonucleotide for each of the discrete reaction sites of the array.

In regard to various methods, each of the discrete reaction sites of the array can have a pitch between the reaction sites in the range of from 50-500 microns and the discrete reaction sites can each have a diameter in the range of from 30-300 microns.

In regard to various methods, each of the discrete reaction sites of the array can have a density of reaction sites in the range of from 50 to 200 reaction sites per mm².

In regard to various methods, the first sub-group can be an array category selected from the group consisting of a row, a column, a diagonal, an even numbered row, an odd numbered row, an even numbered column, an odd numbered column, a first checkerboard pattern, a second checkerboard pattern, and combinations thereof.

In regard to various methods, the second sub-group is an array category selected from the group consisting of a row, a column, a diagonal, an even numbered row, an odd numbered row, an even numbered column, an odd numbered column, a first checkerboard pattern, a second checkerboard pattern, and combinations thereof, wherein the array category of the first sub-group and the second sub-group are different.

In regard to various methods, the first sub-group can be a row, wherein the second unique barcode sequence includes a same sequence within the row of the plurality of arrays and a different sequence between each of the rows of the plurality of arrays.

In regard to various methods, the second sub-group can be the column, wherein the third unique barcode sequence includes a same sequence within the column of the plurality of arrays and a different sequence between each of the columns of the plurality of arrays.

A flow cell configured to capture cells from a biological sample in a hydrogel cage including (a) an inlet configured for receiving a liquid; (b) an outlet configured outputting the liquid; (c) a top layer; (d) a spacer layer having a cut-out portion; and (e) a bottom layer. The top layer and bottom layer are in an opposing relationship with the spacer layer in between the top layer and bottom layer. The top layer, the bottom layer, and the cut-out portion cooperate to form a channel. The inlet and outlet are in liquid communication with the channel allowing liquid flow through the channel. The bottom layer including a plurality of arrays as described herein.

In regard to various flow cells, the plurality of arrays can be arranged in rows and columns.

In regard to various flow cells, each array can include a plurality of discrete sites where a conjugate surface oligonucleotide is coupled to a surface on the bottom layer at each of the discrete sites, the conjugate surface oligonucleotide includes a first oligonucleotide and a second oligonucleotide. The surface can include an uncoated region in between neighboring arrays, the uncoated region comprising an area where no conjugate surface oligonucleotides are coupled to the surface. The first oligonucleotide can include a first unique array barcode sequence for each discrete site location within the array. Each of the first unique array barcode sequences of the first oligonucleotides can corresponds to a mapped discrete site location within the array. The second oligonucleotide can include a second unique barcode sequence. The second unique barcode sequence can include a same sequence within a first sub-group of the plurality of arrays and a different sequence between each of the first sub-groups of the plurality of arrays. Each of the second unique barcode sequences of the second oligonucleotides can correspond to a mapped first sub-group location within the plurality of arrays.

In regard to various flow cells, the second oligonucleotide can further include a random barcode sequence that is a unique sequence for at least 99% of the conjugate surface oligonucleotides.

In regard to various flow cells, the first sub-group can be an array category selected from the group consisting of a row, a column, a diagonal, an even numbered row, an odd numbered row, an even numbered column, an odd numbered column, a first checkerboard pattern, a second checkerboard pattern, and combinations thereof.

In regard to various flow cells, the second sub-group is an array category selected from the group consisting of a row, a column, a diagonal, an even numbered row, an odd numbered row, an even numbered column, an odd numbered column, a first checkerboard pattern, a second checkerboard pattern, and combinations thereof, wherein the array category of the first sub-group and the second sub-group are different.

In regard to various flow cells, the first sub-group can be a row, wherein the second unique barcode sequence includes a same sequence within the row of the plurality of arrays and a different sequence between each of the rows of the plurality of arrays.

In regard to various flow cells, the second sub-group can be the column, wherein the third unique barcode sequence includes a same sequence within the column of the plurality of arrays and a different sequence between each of the columns of the plurality of arrays.

In various embodiments, first surface oligonucleotide complex may be concatenations of first and second oligonucleotides and second surface oligonucleotide complex may be concatenations of the first, second, and third oligonucleotides. That is, a surface oligonucleotide complex can be a partially completed oligonucleotide precursors to a spatial barcode oligonucleotide. In some embodiments, an extension step may be performed to the first or second surface oligonucleotide complex using a ligase to result in a “spatial barcode oligonucleotide.”

As noted above, the term first or second conjugate surface oligonucleotide can refer to a partially completed oligonucleotide precursor to a spatial barcode oligonucleotide.

In some cases, each of the arrays of the plurality are the same. In some cases, an array of the plurality of arrays comprises a pitch between the reaction sites in the range of 50-500 microns, and wherein the reaction sites each have a diameter in the range of 30-300 microns. In some cases, an array of the plurality of arrays comprises a density of reaction sites in the range of from 50 to 200 reaction sites per mm².

In various embodiments, the first oligonucleotide can include a first barcode segment (BC₁ or 106) and a first sequence (S₁). The first barcode segment can include a unique array barcode sequence. In various embodiments, the second oligonucleotide can include a second barcode segment (BC₂) and a second sequence (S₂). The second barcode segment can include a second unique barcode sequence. In various embodiments, the third oligonucleotide can include a third barcode segment (BC₃) and third sequence (S₃). The third barcode segment can include a third unique barcode sequence.

### BRIEF DESCRIPTIONS OF THE DRAWINGS

FIG. 1A illustrates one format of a combinatorial spatial barcode which may be used with the systems and methods described herein.

FIG. 1B illustrates an embodiment of the systems and methods described herein in which a final barcode segment is attached by tagmentation.

FIGS. 2A and 2B illustrate an embodiment for producing a spatially barcoded surface.

FIGS. 2C-2E illustrate embodiments for producing spatially barcoded surfaces in which channels are partially coincident with arrays of first oligonucleotide either by adjusting the spot pattern of the arrays (FIG. 2D), employing multiple channel templates that off-set channel positions (FIG. 2E).

FIG. 2F illustrates the production of combinatorial barcodes in one dimension by employing a plurality of channel templates with different channel widths and off-sets.

FIG. 3 illustrates an appliance for creating channels for applying reagents to rows or columns of spotted arrays on a surface.

FIG. 4A illustrates a top view of the bottom layer of a flow cell without any spatial barcoded oligonucleotides.

FIG. 4B illustrates a top view of the spacer layer with a cut-out region suitable for use as part of the flow cell.

FIG. 4C illustrates a top view of the top layer of the flow cell where the top layer has an inlet and outlet opening.

FIG. 5 illustrates a top view of the bottom layer and spacer letter laminated together along with a plurality of arrays containing barcoded oligonucleotides.

FIG. 6 illustrates an exploded top view of a single array having a plurality of overlapping droplets that partly extends beyond the boundary of the single array.

### DETAILED DESCRIPTION

The practice of the systems and methods described herein may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, monoclonal antibodies, antibody display systems, cell and tissue culture techniques, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; Retroviruses; and Molecular Cloning: A Laboratory Manual (all from Cold Spring Harbor Laboratory Press); Renault and Duchateau, Editors, Site-directed Insertion of Transgenes (Springer, Heidelberg, 2013); Lutz and Bornscheuer, Editors, Protein Engineering Handbook (Wiley-VCH, 2009); and the like. Guidance for selecting materials and components to carry out particular functions may be found in available treatises and references on scientific instrumentation including, but not limited to, Moore et al, Building Scientific Apparatus, Third Edition (Perseus Books, Cambridge, MA); Hermanson, Bioconjugate Techniques, 3rd Edition (Academic Press, 2013); and like references.

The systems and methods described herein are directed to making or generating spatially barcoded surfaces and their use to analyze molecules, especially nucleic acid molecules, of biological cells disposed on such surfaces. The systems and methods described herein are also directed to spatially barcoding nucleic acid molecules disposed or captured on a surface. Spatial barcodes may be combinatorial in the sense that each barcode is a combination of at least three segments: two segments that identify the position of an array on the surface and a third segment that identifies the position of the barcode oligonucleotide, or the nucleic acid molecule it is attached to, within the array. Moreover, in some embodiments, the final library of spatial barcodes comprises every combination of the possible sequences of the first, second and third barcode segments. Thus, in embodiments employing three barcode segments, the number of first oligonucleotides (each containing a first barcode segment) in an array, the number of channels for delivering second oligonucleotides (each containing a second barcode segment) and the number of channels for delivering third oligonucleotides (each containing a third barcode segment) determines the total number of different barcodes on a surface. For example, for an array of 384 first oligonucleotides, a first channel template of 50 channels and a second channel template of 50 channels, a surface may have 980,000 (=50×50×384) different barcodes. Channel templates and gaskets to sealingly attach templates to a surface may be made using fabrication techniques employed for microfluidics devices.

A wide variety of surfaces may be used with the systems and methods described herein. In some embodiments, surfaces are two-dimensional planar surfaces of a solid support material. Such solid support materials may comprise non-porous solids that may be derivatized with conventional functionalities by which oligonucleotides may be attached (e.g. Devor et al, Integrated DNA Technologies (2005), or the like). In some embodiments, such solid support materials may comprise glass, plastic, silicon, metal oxides, or the like. In some embodiments, a surface is a glass support material, such as a glass slide.

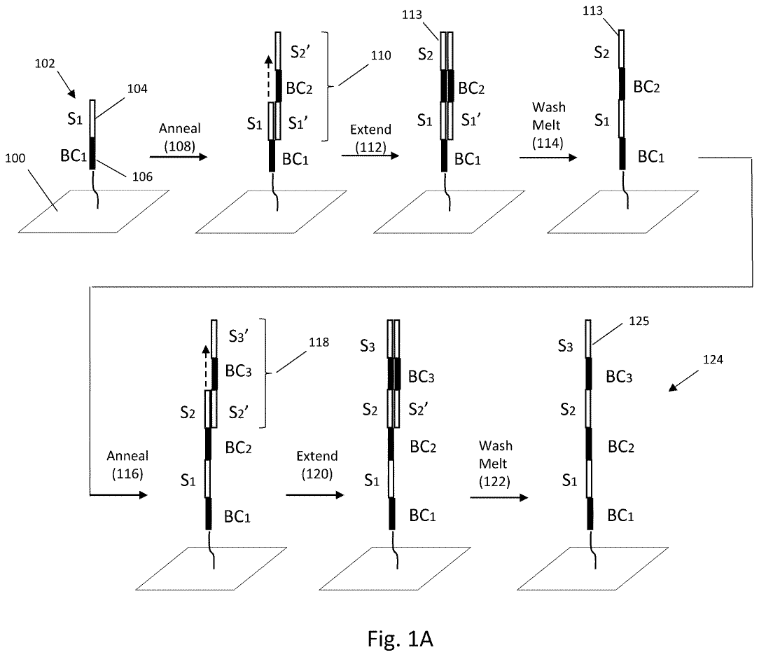

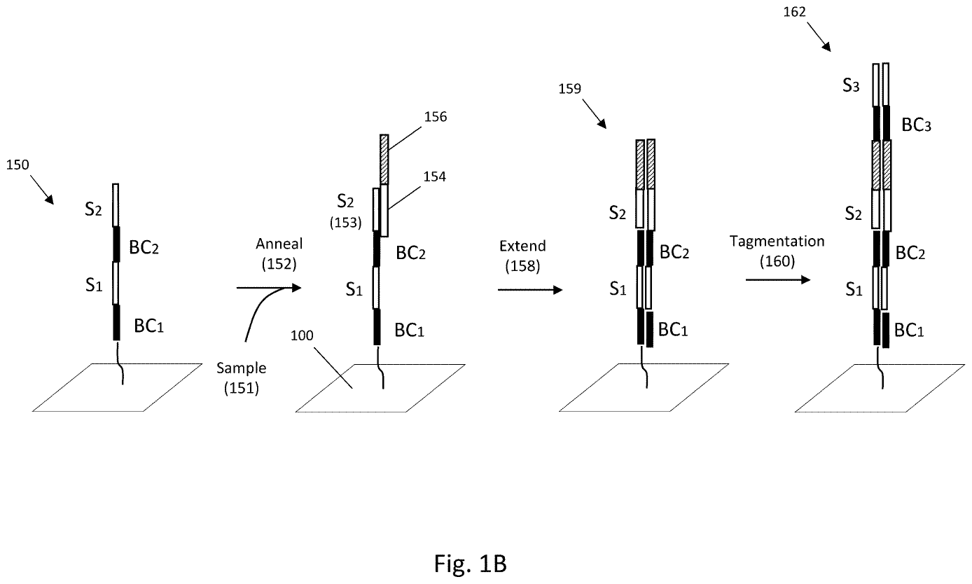

In various embodiments of the systems and methods described herein, barcode segments may be attached before and/or after capture and replication of nucleic acid molecules from samples. In other words, the order in which barcode segments and sample nucleic acids are assembled on a surface may vary so that the ordering of cDNA (transcribed from a captured nucleic acid) and the barcode segments making up a spatial barcode may be selected. In different embodiments, such ordering (from the surface) may be as follows: -cDNA-BC₁-BC₂-BC₃; BC₁-cDNA-BC₂-BC₃; BC₁-BC₂-cDNA-BC₃; or BC₁-BC₂-BC₃-cDNA, where BC₁, BC₂ and BC₃ represent the first, second and third oligonucleotides (containing the first, second and third barcode segments), respectively. The assembly of first, second and third oligonucleotides to produce a barcoded surface or the assembly of cDNAs, and first, second and third oligonucleotides to produce a surface with spatially barcoded cDNAs is accomplished using conventional methods for linking nucleic acid molecules to one another or to surfaces, which are exemplified for the embodiments described in FIGS. 1A and 1B.

Although embodiments are disclosed showing the formation of spatial barcodes comprising two or three barcode segments, the systems and methods described herein may also include combinatorial spatial barcodes of a plurality of barcode segments. In some embodiments, combinatorial spatial barcodes comprise from 3 to 6 barcode segments; or from 3 to 5 segments; or from 3 to 4 segments. In some embodiments, combinatorial spatial barcodes having greater than three barcode segments may be produced by applying additional steps of partitioning and reacting using (or reusing) channel templates loaded with oligonucleotides comprising different combinations of barcode sequences.

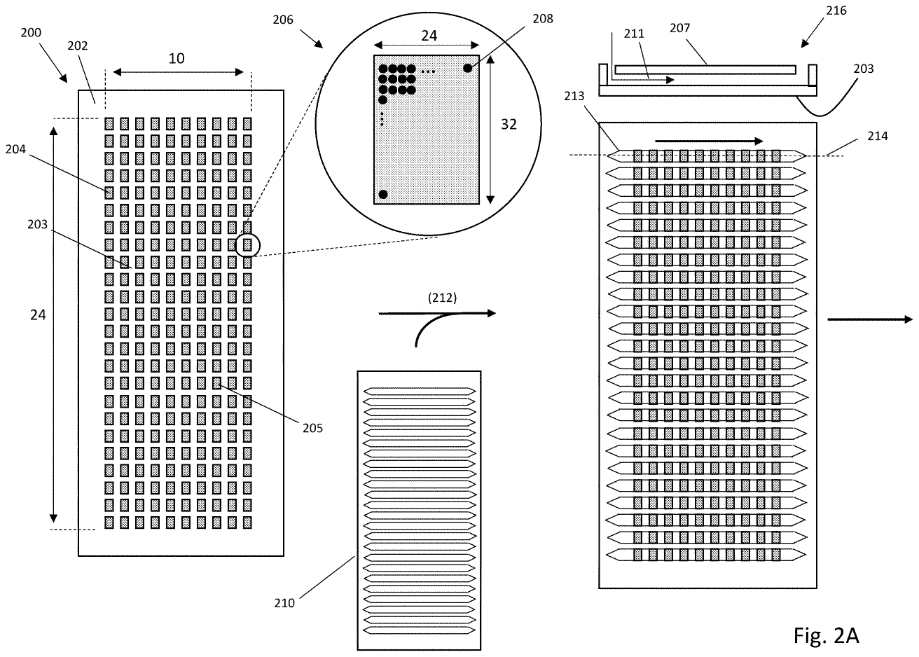

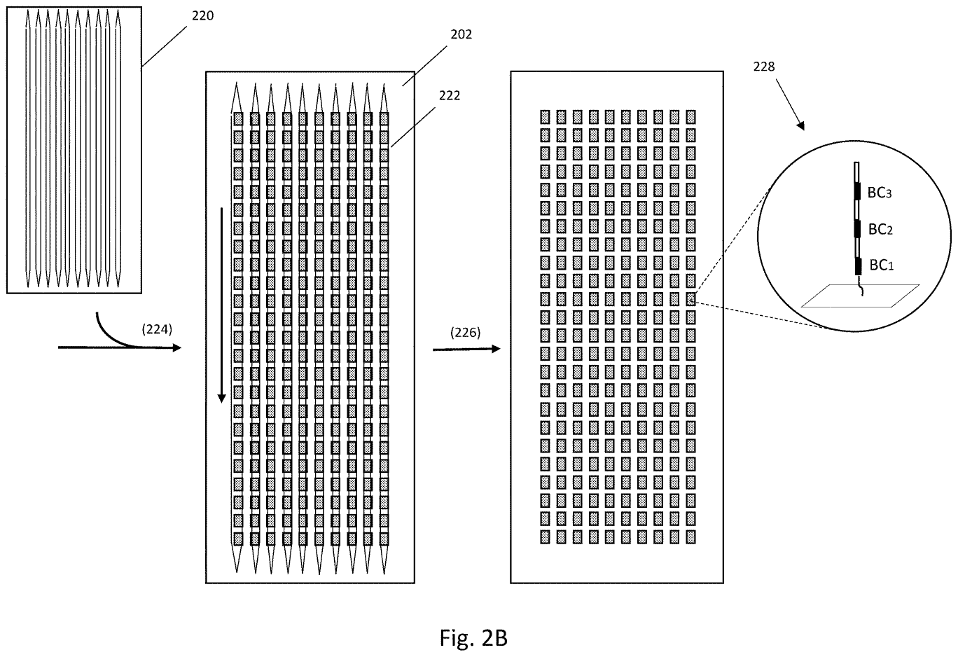

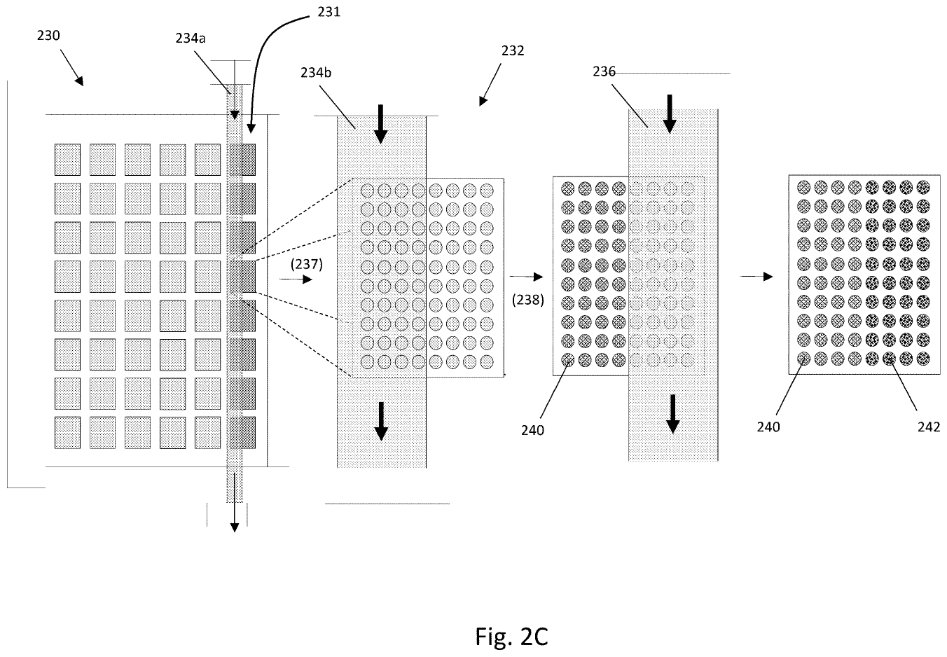

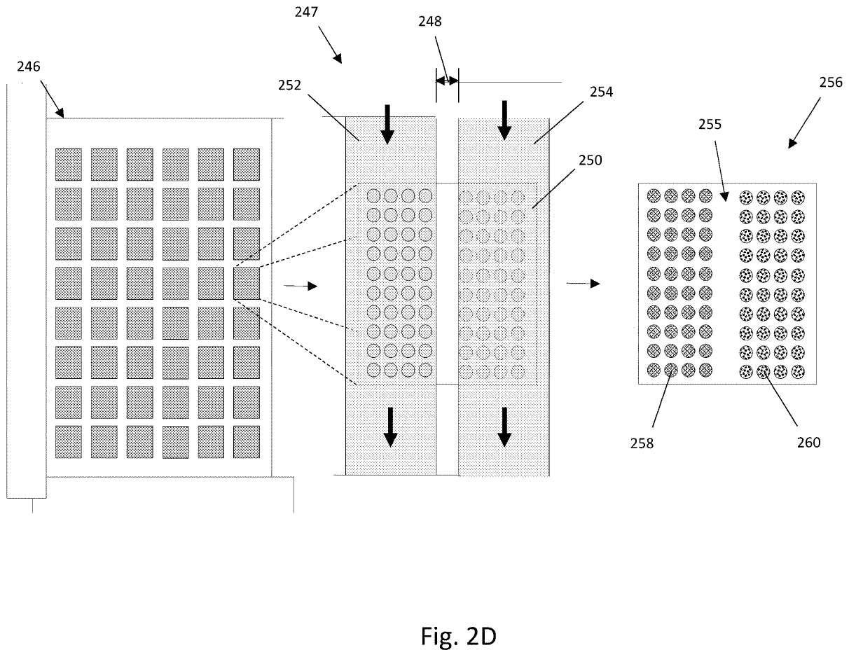

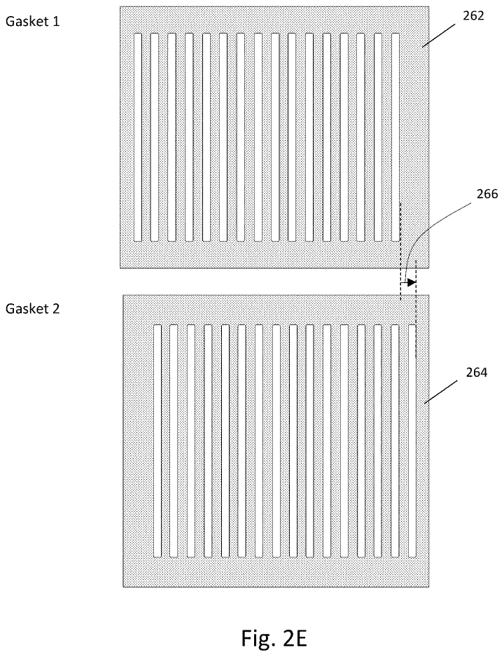

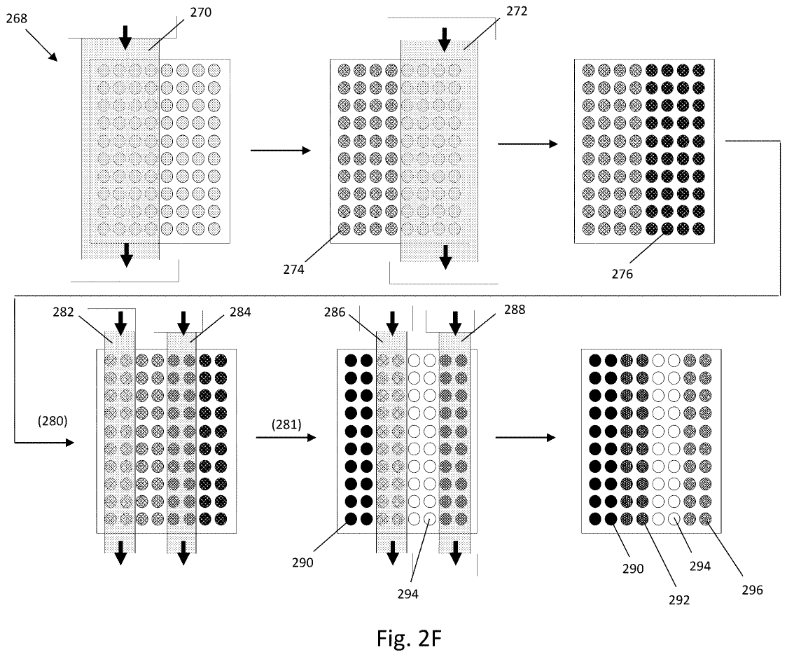

In some embodiments, an array of first oligonucleotide arrays is synthesized (or disposed) on a surface, e.g. as illustrated in FIG. 2A, after which second and third oligonucleotides are attached by forming orthogonal channels for delivering the oligonucleotides (e.g. as illustrated in FIGS. 2A-2B). In some embodiments, surface (202) (see FIG. 2A) may be free of capture oligonucleotides so that the interstitial space (203) between arrays (and between spots or reaction sites within arrays) are free of barcodes. In other embodiments, surface (202) may be coated with capture oligonucleotides for capturing the various barcode oligonucleotides (first, second or third), which may be followed by either extension or ligation to form a combinatorial barcode. In other words, in some embodiments, surface functionalities may comprise capture oligonucleotides. In such latter embodiments, barcoded surfaces may be produced wherein the interstitial spaces (e.g. 203) in an array of arrays contain barcodes of one or more segments. In some embodiments, the ordering of channel delivery and droplet delivery of the first, second and third oligonucleotides may differ. In some embodiments, an array of array of first oligonucleotides is delivered by droplets, followed by channel delivery of second oligonucleotides and third oligonucleotides. In other embodiments, first oligonucleotides are delivered by channel, an array of arrays of second oligonucleotides is delivered by droplets, and third oligonucleotides are delivered by channel. In still other embodiments, first oligonucleotides are delivered by channel, second oligonucleotides are delivered by channel, and an array of arrays of third oligonucleotides is delivered by droplets.

FIG. 1A illustrates one embodiment for sequentially linking three barcode segments to form a spatial barcode for a surface, after which a sample nucleic acid may be captured (i.e. the fourth format described above: BC₁-BC₂-BC₃-sample NA). In one embodiment, first oligonucleotide (102) comprising first barcode segment (BC₁)(106) and sequence (S₁)(104) is attached to surface (100) by its 5′ end by any of a variety of linkages well-known to those skilled in the art, e.g. Beaucage, Curr. Med. Chem., 8(10): 1213-1244 (2001); Frydrych-Tomczak et al, BioTechnologia, 95(1): 5-16 (2014); Ratajczak et al, Methods Mol. Biol., 1368: 25-36 (2016); Uszczynska et al, LabChip, 12(6): 1151-1156 (2012): and the like. Such linkages are formed by reaction of a surface functionality and a complementary functionality of the oligonucleotide being attached. In some embodiments, oligonucleotides being attached, such as, capture oligonucleotides or barcode oligonucleotides, are attached by their 5′ ends, for example, so that their 3′ ends remain free for later extension by a polymerase.

In accordance with some embodiments, first oligonucleotides (102) having different barcode sequences are delivered to separate known locations in an array using a DNA printing device, such as a device manufactured by M2 Automation (Berlin, Germany), Scienion (Berlin, Germany), or the like. In some embodiments, inkjet delivery systems may be used to construct the plurality of arrays, e.g. Cartesian Technologies (Irvine, CA); Barczak et al, Genome Research, 13: 1775-1785 (2003); and the like. In some embodiments, first oligonucleotides of the plurality of arrays may be synthesized in situ using a variety of array synthesis technologies, e.g. Singh-Gasson et al, Nature Biotechnology, 17: 974-978 (1999); Horgan et al, International patent application WO2022/013094; Le, Recent Progress in Ink Jet Technologies II, chapter 1 (1999); Hughes et al, Nature Biotechnology, 19: 342-347 (2001); and the like. In some embodiments, such arrays comprise spatially compact rectilinear or hexagonal arrays of non-overlapping, i.e. spatially discrete, reaction sites substantially uniformly coated with first oligonucleotides (102). In some embodiments, arrays of such reaction sites may have, but are not limited to, pitches (center-to-center distances) in the range of from 50-500 μm and diameters in the range of from 30-100 μm. Returning to FIG. 1A, the first oligonucleotides (102) attached to surface (100) can be hybridized (or annealed (108)) to second oligonucleotides (110) comprising segments S₁′ (complementary to segment S₁ (104)), second barcode segment BC₂, and segment S₂′. Afterwards, reagents may be introduced to extend first oligonucleotide (102) so that BC₂ and S₂′ of second oligonucleotide (110) are copied to form a first conjugate surface oligonucleotide. In alternative embodiments, second barcode segment (113) may be attached to first oligonucleotide (102) by ligating a second oligonucleotide using, for example, a ligase, to thereby form a first conjugate surface oligonucleotide. In some alternative embodiments, successive oligonucleotide segments may be attached by ligation using a ligase and splint oligonucleotides that form a duplex with the two oligonucleotides to be ligated. In further embodiments, successive oligonucleotide segments may be attached by ligation using a circligase.

A method of delivering second oligonucleotides (110) and reagents for extending first oligonucleotides (102) is illustrated in FIGS. 2A-2B. After hybridization and extension (112), hybridized and copied second oligonucleotide (110) can be melted (114) from strand (113). Strand (113) (sometimes referred to herein as the “first conjugate surface oligonucleotide”) can be annealed (116) to third oligonucleotide (118) comprising segment S₂′ (complementary to segment S₂ of strand (113)), third barcode segment (BC₃) and segment S₃′. After extension (120) and washing and melting (122), the result is spatial barcode (124) comprising barcode segments BC₁, BC₂ and BC₃, the combination of which may be unique for each reaction site in the plurality of arrays. In some embodiments, segment S₃ (125) may serve as a capture oligonucleotide. For example, it may be a polyT sequence for capturing polyA-tailed messenger RNAs from cells of a sample being analyzed on surface (100). Similar to above, in alternative embodiments, third barcode segment (118) may be attached to strand (113) by ligation.

FIG. 1B illustrates an alternative embodiment that employs tagmentation to attach a third barcode component (thereby forming a barcoded sample nucleic acid of the third format above, namely: BC₁-BC₂-sample NA-BC₃. A review of the tagmentation technique is given in Adey, Genome Research, 31: 1693-1705 (2021); U.S. Pat. Nos. 9,115,396; 9,085,801; 11,319,534; and the like, which are incorporated herein by reference. Barcode segments BC₁-S₁ and BC₂-S₂ (150) can be assembled as described in FIG. 1A where segment S₂ is a capture probe (for example, a polyT segment specific for polyA messenger RNA of a biological sample). Biological sample (151) can be contacted with surface (100) so that polyA mRNA contained therein anneals (152) to capture oligonucleotides (S₂, 153), wherein the mRNA comprises polyA segment (154) and coding segment (156). After extension with a reverse transcriptase and optional template switching, double stranded structure (159) may be obtained, after which it is subjected to tagmentation (160) to attach final barcode segment, BC₃, to give final sequence (162). Using similar procedures, each of the formats (-sample NA-BC₁-BC₂-BC₃; -BC₁-sample NA-BC₂-BC₃; -BC₁-BC₂-sample NA-BC₃; or -BC₁-BC₂-BC₃-sample NA) may be synthesized.

In accordance with some embodiments described herein, second and third oligonucleotides comprising second and third barcode segments, respectively, are delivered to the plurality of arrays by channels as illustrated in FIGS. 2A-2B. Alternative embodiments for delivering and conjugating to first oligonucleotides (or the conjugates of first and second oligonucleotides) may comprise the use of photo-masks and photo-activated ligation, for example, as taught by van Dam, Thesis (California Institute of Technology, 2005). As shown in FIG. 2A, a plurality of arrays (e.g. 204) can be synthesized on surface (202) of slide, or substrate, (200). In this illustration, the plurality of arrays is 240, arranged in a 24×10 rectilinear format. The spacing of the arrays on surface (202) is exaggerated for the sake of illustration. Blow-up (206) of an array shows a 32×24 array of reaction sites (208). In some embodiments, each array of the plurality has the same first oligonucleotides in the same positions. Thus, for example, the sequence of the barcode segment of first oligonucleotide at row 18 and column 11 of array (205) is the same as that of the first oligonucleotide at row 18 and column 11 of array (204). That is, in some embodiments, each of the arrays of a plurality comprise the same first oligonucleotides.

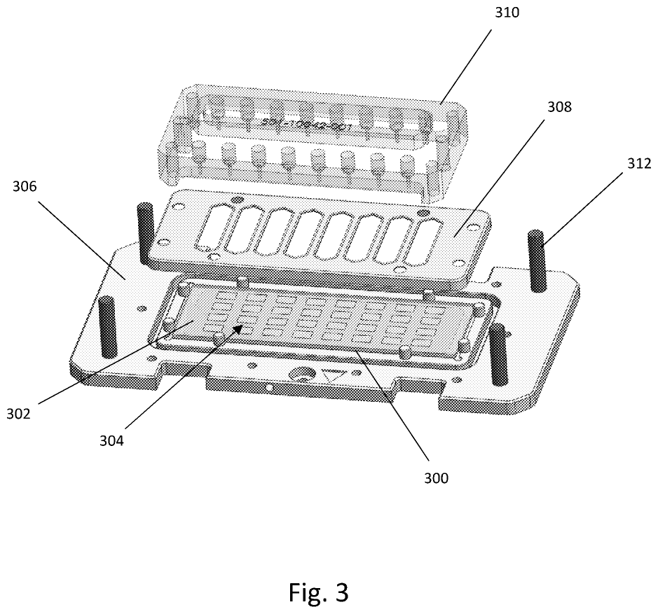

In this embodiment, second oligonucleotides and associated extension reagents (for example, DNA polymerases, reaction buffers, dNTPs, and so on) are delivered by way of channels formed in a layer of material (for example, an elastomeric plastic, or the like), forming a channel body or template that can be placed over the plurality of arrays and partitions it into a plurality of rows or a plurality of columns. One of ordinary skill in the art would understand that the pluralities of arrays, rows, columns, first channels, second channels, and the like, are independent quantities; that is, the values of the pluralities for these separate features need not be the same in any particular embodiment. As illustrated in FIG. 2A, channel template (210) can be placed (212) on surface (202) to partition the plurality of arrays into a plurality of 24 rows of 10 arrays each. Placement of channel template (210) on surface (202) may be implemented using a simple appliance similar to that illustrated in FIG. 3, which sandwiches channel template (210) between surface (202) of substrate (203) and cover (207). Channel templates may vary widely in design and composition depending on the magnitude and arrangement of a plurality of arrays, the size and arrangement of arrays of reaction sites, and the methods used to couple first, second and third oligonucleotides. Channel templates may be fabricated from wide variety of materials well-known in the microfluidics field, such as, silicon, glass, plastic, or the like, e.g. Ren et al, Acc. Chem. Res., 46(11): 2396-2406 (2013). In some embodiments, channel templates may comprise a plastic, such as, polystyrene, polyethylenetetraphthalate glycol, polyethylene terephthalate, polymethylmethacrylate, polyvinylchloride, polycarbonate, thermo plastic elastomer or the like. Guidance in the selection of plastics and fabrication methodologies may be found in the following references: Becker et al, Talanta, 56: 267-287 (2002); Fiorini et al, Biotechniques, 38(3): 429-446 (2005); Bjornson et al, U.S. Pat. No. 6,803,019; Soane et al, U.S. Pat. No. 6,176,962; Schaevitz et al, U.S. Pat. No. 6,908,594; Neyer et al, U.S. Pat. No. 6,838,156; and the like, which references are incorporated herein by reference.

As illustrated in cross-sectional view (216), along median (214) of channel (213), after assembly of substrate (203), channel template (210) and cover (207) exclusive flow paths (211) are created for each row of arrays. Thus, each array of a given row may receive the same second oligonucleotide. In some embodiments, the sequence of the barcode segment of each second oligonucleotide of a different row is different, so that the sequence of second barcode segments uniquely identifies the row on which a spatial barcode is located.

After second oligonucleotides are delivered and coupled to first oligonucleotides, row channel template (210) can be removed. As illustrated in FIG. 2B, column channel template (220) can be placed (224) on surface (202) of substrate (203) to partition the plurality of arrays into a plurality of 10 columns (e.g. 222) of 24 arrays each. As with the partition into rows, channel template (220) may create an exclusive flow path for each column, which permits the arrays of each column to be exposed to the same third oligonucleotide. In some embodiments, the sequence of the barcode segment of each third oligonucleotide of a different column is different, so that the sequence of third barcode segments uniquely identifies the column on which a spatial barcode is located. After coupling of the third oligonucleotide (226), a spatially barcoded surface can be created with spatial barcodes of the form shown in blow-up (228).

In some embodiments, the number of unique barcodes on a surface may be increased by providing channels that are coincident with subsets of reaction sites of the rows or columns of arrays. FIG. 2C illustrates an example of this embodiment for the columns of array (232) shown in a blow-up view with respect to array of arrays (230). In this embodiment, widths of channels (e.g. 234a and 234b in blow-up) are fabricated so that the channels are coincident with half of the spots (or reaction sites) of the arrays of column (231, darker shaded sub-arrays), so that barcode oligonucleotides may be attached (237) sequentially to a first half of reaction sites (e.g. 240) and then (238) to a second half of reaction sites (e.g. 242). This may be accomplished by using two different channel templates with channel positions off-set by (for example) half an array width, or by moving a single channel template a half array width. FIG. 2E illustrates the case wherein two channel templates (262 and 264) (or the gasket components of such channel templates) are used with channels off-set by predetermined amount (266) so that different reaction sites are exposed to reagents delivered by the channels. After such steps, array of arrays (230) will have twice the number of different spatial barcodes than embodiments in which each entire sub-array (e.g. 232) is coincident with the channels delivering the barcode oligonucleotides. In some embodiments, such as shown in FIG. 2D, channel templates may be fabricated so that the halves of the sub-arrays are coincident with different channels, wherein space (248) is selected so that a wall of the channel template can be fitted without covering or obstructing reaction sites. This configuration increases the speed of fabrication and simplifies the oligonucleotide deposition process, so that, for example, different barcode segments are simultaneously added to each half of reaction sites giving products (258 and 260) in a single sub-array (256). This embodiment is facilitated by forming sub-arrays with gap (255) in sub-array (256) which separates the halves and provides space for the wall of the channel template. In other embodiments, sub-arrays may be formed with multiple gaps, for example, dividing reaction sites into three regions instead of two, and such gaps may be formed in both horizontal and vertical directions, for example, for the attachment of second and third oligonucleotides, respectively.

In some embodiments, as illustrated in FIG. 2F, spatial barcode oligonucleotides may be formed combinatorially by delivering their components with channels of different widths. For example, channel (270) may be established by attaching a first channel template to a surface comprising sub-array (268), which delivers barcode oligonucleotides which react with either the surface or a conjugate surface oligonucleotide to give product (e.g. 274) in half of the reaction sites of sub-array (268). Subsequently, channel (272) may be established by attaching a second channel template which delivers barcode oligonucleotides which react with either the surface or a conjugate surface oligonucleotide to give product (e.g. 276) in the other half of the reaction sites of sub-array (268). Next, channels (282 and 284) are established by attaching a third channel template with narrower spaced apart channels that deliver (280) reagents to a first and third quadrants of reaction sites of sub-array (268), which after reacting give products (290) and (294). After step (280), channels (286 and 288) are established by attaching a fourth channel template with spaced apart channels that deliver (281) reagent to a second and fourth quadrants of reaction sites of sub-array (268), which after reacting give products (292 and 296). In such embodiments, barcoded surfaces with four times the number of unique barcodes may be produced as compared to embodiments wherein channels are coincident with entire sub-arrays.

As mentioned above, channel templates (210) and (220) may be applied to surface (202) using an appliance as illustrated in FIG. 3, or like apparatus. Substrate (300) comprising surface (302) with plurality of arrays (304) can be placed in base (306) and channel template (308) can be placed on top creating a partition of rows (or columns) of arrays. On top of channel template (308), manifold (310) may be placed for providing conduits from reagent reservoirs or plates to the channels created by channel template (308). Finally, a top plate may be aligned by alignment pins (312) and places on top to complete the assembly.

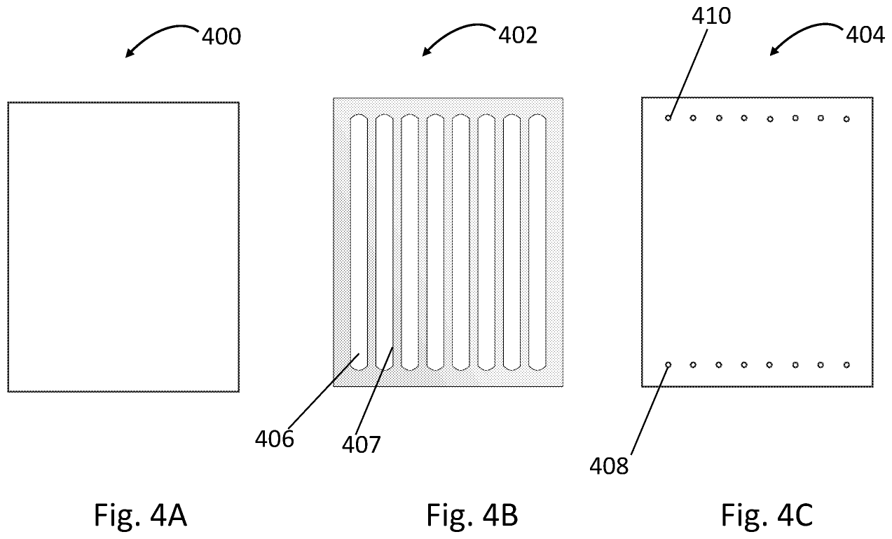

As described herein, a sample fluid containing cells can be analyzed in a flow cell that includes a bottom layer (400), a spacer layer (402), and top layer (404) (see FIG. 4A, FIG. 4B, and FIG. 4C, respectively). The bottom layer (400) and the top layer (404) can both be a glass or plastic material. The spacer layer (402) can be a double-sided pressure sensitive adhesive with a cut-out portion (406). Referring back to FIG. 4B, a peripheral portion (407) provides a boundary for the cut-out portion (406). In various embodiments, spacer layer (402) can include a core PET layer with a pressure sensitive adhesive coating on both sides of the PET layer. Top layer (404) includes an inlet (408) configured to receive a liquid and an outlet (410) configured to output the liquid. In various embodiments, the inlet (408) and outlet (410) can both be represented as through holes in the top layer (404).

In various embodiments, a row can be three or more arrays arranged in an approximately linear line (e.g., horizontal) that runs approximately perpendicular to a liquid flow in a channel. A column can be three or more arrays arranged in an approximately linear line (e.g., vertical) that runs approximately parallel to the liquid flow in the channel. In various embodiments, the approximately linear line for the row is about orthogonal to the approximately linear line for the column.

To form a flow cell, the top layer (404) and bottom layer (400) are in an opposing relationship with the spacer layer (402) in between the top layer (404) and bottom layer (400). The top layer (404), the bottom layer (400), and the cut-out portion (406) cooperate to form a channel, wherein the inlet (408) and outlet (410) are in liquid communication with the channel allowing liquid flow through the channel. The channel can have a height defined by the height of the spacer layer (402) and an area defined by the cut-out portion (406). The peripheral portion (407) of the spacer layer (402) forms the walls of the channel. The bottom layer (400) forms the floor of the channel and the top layer (404) forms the roof of the channel. The spacer layer (402) of FIG. 4B includes eight cut-out portions (406). The top layer (404) of FIG. 4C includes 8 inlets and 8 outlets that are arranged at opposing ends of the cut-portions (406) when laminated.

In various embodiments, the bottom layer (400) can have a spatial barcode surface. A cell can be captured by a hydrogel cage and at the same time enclose a portion of the bottom layer that has a discrete area containing the particular spatial barcode. This particular spatial barcode can contain information such as the location of the captured cell in the hydrogel cage within the flow cell. A spatial barcode can be referred to as a surface oligonucleotide complex that include a first oligonucleotide, a second oligonucleotide, and/or a third oligonucleotide. The various oligonucleotide of the spatial barcode can be coupled together as a composite oligonucleotide.

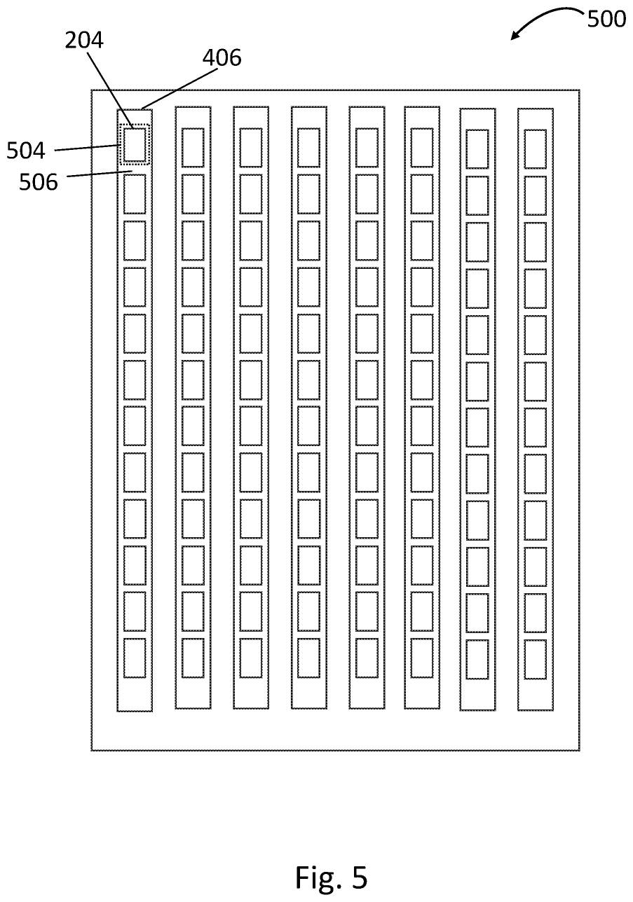

In various embodiments, bottom layer can be synthesized with a barcoded surface in the form of a plurality of arrays. Subsequent to the synthesis, the spacer layer can be laminated on the bottom layer with the barcoded surface as illustrated in FIG. 5. The bottom layer and spacer layer laminated together form a composite partial assembly (500) as illustrated in FIG. 5. In addition, FIG. 5 shows that the plurality of arrays (204) are arranged such that the columns of the array are coincident with the channel as defined by the cut-out portion (406). In various embodiments, the area of the array can range from about 5 mm² to about 200 mm². For example, the array can each have a dimension of 5 mm×8 mm or an area of 40 mm².

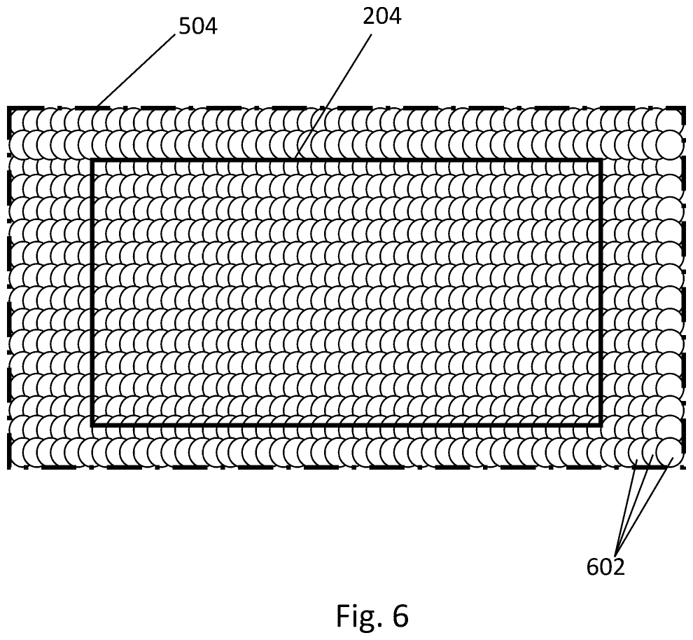

In an alternative embodiment, a second oligonucleotide and/or a third oligonucleotide can be coupled to the first oligonucleotide without using a channel template (e.g., 210 or 220). For instance, a liquid containing the second oligonucleotide can be disposed on each array along a row instead of flowing the liquid through a temporary flow channel along the row. FIG. 6 illustrates an example of a single array (204) having a plurality of overlapping droplets 602 that partly extends beyond the boundary of the single array. The overlapping droplets 602 can coalesce to form a single puddle 504 of liquid with an indicated boundary of a rectangular dot dash line that is larger than the array (204) with an indicated boundary of a smaller rectangular solid line. However, the single puddle does not touch another puddle coating a neighboring array so that there is an uncoated region or interstitial region 506, as indicated in FIG. 5.

A method of making a barcoded surface includes synthesizing a plurality of arrays (204) arranged in rows (e.g., 24) and columns (e.g., 10) as illustrated in FIG. 2A. In various embodiments, the rows and the columns of the plurality of arrays can be arranged in an orthogonal relationship, as illustrated in FIG. 2A. Each array can include a plurality of discrete reaction sites (208) as illustrated in blow up portion (206) of FIG. 2A. For example, the array can be formed of a matrix of 24×32 discrete reaction sites where each reaction site has a first oligonucleotide with a unique array barcode that corresponds to a location within the array. In various embodiments, each of the discrete reaction sites of the array are formed from a disposed droplet containing the first oligonucleotide that includes a unique array barcode sequence.

In various embodiments, the unique array barcode sequence of the first oligonucleotides represents a series of nucleotides having a code that is mapped to particular discrete reaction site within the array. In the process of making the discrete reaction sites, a separate liquid solution can be prepared for each unique array barcode of the oligonucleotides. For example, a matrix of 24×32 discrete reaction sites can have 768 separate solutions prepared for the first oligonucleotide where each one has a unique array barcode that is mapped to a particular discrete reaction location. In various embodiments, each of the discrete reaction sites can have a pitch between the reaction sites in the range of from 50-500 microns and each of the discrete reaction sites can have a diameter in the range of from 30-300 microns. In various embodiments, each of the discrete reaction sites of the array can have a density of reaction sites in the range of from 50 to 200 discrete reaction sites per mm².

The surface of the solid support can include a coupling layer that is coupled to the surface. The coupling layer can include a silane reactive moiety that binds to a silanol group on the surface and amine reactive moiety. The first oligonucleotide can be derivatized to have an amine group proximate to the 5′ end. In various embodiments, the synthesizing the plurality of arrays includes coupling the amine moiety of the first oligonucleotide with the surface via the coupling layer, wherein the coupling layer includes the amine reactive moiety. In various embodiments, each discrete reaction site includes a first oligonucleotide (102) coupled to the surface (100) of a solid support at a 5′ end of the first oligonucleotide (see FIG. 1A). It is worthwhile to note that the discrete reaction site is configured to react or bind with another oligonucleotide in subsequent processes. In various embodiments, the surface comprises a glass surface and the coupling layer comprises a silane linkage to the glass surface, and the amine reactive moiety comprises a N-hydroxysuccinimide moiety. In various embodiments, the coupling layer can be attached to the surface and have a reactive group such as, for example, an alkyne moiety, an azide moiety, and/or amine moiety. The alkyne moiety of the coupling layer can react with an azide moiety that is coupled to the first oligonucleotide. The azide moiety of the coupling layer can react with an alkyne moiety of (dibenzocyclooctyne) DBCO moiety that is coupled to the first oligonucleotide. The amine moiety of the coupling layer can react with a N-hydroxysuccinimide moiety that is coupled to the first oligonucleotide.

The plurality of arrays can be constructed such that the surface includes an uncoated region (506) in between neighboring arrays (204), as illustrated in FIG. 5. The uncoated region (506) includes an area where no first oligonucleotides are coupled to the surface. In various embodiments, the arrays (204) as illustrated in FIG. 2A or FIG. 5 can be the same where each array has a same patterned location of the unique array barcode sequences for the first oligonucleotides for each of the discrete reaction sites of the array. Thus, the deposition process for the discrete reaction sites can be the same for each of the arrays (204) where the array can be formed in a 12 row×8 column format as illustrated in FIG. 5. In various embodiments, the unique array barcode sequence of the first oligonucleotide provides the mapped location within the array, but not the row and/or column location of the plurality of arrays.

Now that the first oligonucleotide (102) has been attached to the surface to form a plurality of arrays, a second oligonucleotide can be coupled to the first oligonucleotide (110) by an annealing step (108), as shown in FIG. 1A. A liquid containing a second oligonucleotide can be disposed onto each array (204) of the plurality of arrays where a portion of the uncoated region in between neighboring arrays is not covered by the liquid containing the second oligonucleotide. FIG. 5 shows an example of a puddle 504 of the liquid containing the second oligonucleotide that coats array (204). A dotted line is used to show the boundary of puddle 504 in FIG. 5.

In various embodiments, the liquid containing the second oligonucleotide can be disposed onto each of the arrays so that there is a plurality of puddles, but in way where the neighboring puddles do not touch each other. Each puddle can be in the form of small single pool of liquid. In various embodiments, the puddle can be formed by disposing the liquid as a plurality of dispensed droplets that merge together. For example, the droplets can be formed from an ink jet nozzle or piezoelectric pump. The plurality of dispensed droplets can have a pitch along the x axis ranging from about 1 micron to about 40 microns, and a pitch along the y axis ranging from about 1 micron to about 40 microns. The droplets can have a diameter ranging from about 80 microns to about 140 microns, and a volume ranging from about 200 picoliters to about 350 picoliters. In various embodiments, a piezoelectric pump can dispense droplets at a rate of 1 to about 500 drops/second, and preferably at 2 to about 50 drops/second. A dispensing pipetter of a piezoelectric pump can travel at a rate of 10,000 to 500,000 microns/sec, and preferably at 100,000 to 300,000 microns/sec while dispensing liquid.

A plurality of puddles can be incubated on the surface for a predetermined period of time ranging from 10 minutes to 24 hours and preferably ranging from 10 minutes to 3 hours. During this incubation, the humidity can be controlled to range from about 50% RH to about 90% RH. After incubation with the second oligonucleotide, the liquid containing the second oligonucleotide can be washed off. It should be noted that not coating all of the interstitial regions in between the arrays, a lower volume of the liquid containing the second oligonucleotide is used for deposition compared the processes that uses a channel template. Under various circumstances, it is advantageous to reduce the volume of a liquid containing custom made oligonucleotide so as to reduce the expense of manufacturing. Instrumentation used for liquid deposition can require about 20 to 30 microliters of reagent to cover 8 arrays in a single row which is about 10 time less than the amount of liquid needed to flow through a row channel template. In addition, the time required to coat a row of the plurality of the array using puddles was about 5 to 7 minutes, which is significantly less than the time required to coat a row of the plurality of the array using the row channel template.

Each puddle can be in the form of small single pool of liquid. In various embodiments, the puddle can be formed by disposing the liquid as a plurality of dispensed droplets that merge together. For example, the droplets can be formed from an ink jet nozzle or piezoelectric pump. The plurality of dispensed droplets can have a pitch along the x axis ranging from about 1 micron to about 40 microns, and a pitch along the y axis ranging from about 1 micron to about 40 microns. The droplets can have a diameter ranging from about 80 microns to about 140 microns, and a volume ranging from about 200 picoliters to about 350 picoliters.

In various embodiments, the liquid containing the second oligonucleotide can be disposed as a single droplet onto each of the discrete reaction sites of the arrays, wherein each droplet at least partly covers or fully covers each of the discrete reaction sites. The droplets can be dispensed in a way that neighboring droplets of the neighboring discrete reaction sites do not touch.

In another embodiment, the liquid containing the second oligonucleotide can be disposed as a single droplet onto each of the discrete reaction sites of the arrays, wherein each droplet fully covers each of the discrete reaction sites and partly covers a portion of the interstitial region in between discrete reaction sites. However, the droplets that overcoat the discrete reaction sites are dispensed in a way that neighboring droplets of the neighboring discrete reaction sites do not touch.

In various embodiments, the second oligonucleotide couples to the first oligonucleotide to form a first surface oligonucleotide complex. The process of forming the first surface oligonucleotide complex can also be referred to as hybridizing or annealing. The second oligonucleotide includes a unique row barcode sequence that has the same sequence within a row and a different sequence between each of the rows of the plurality of arrays. Each of the unique row barcode sequences of the second oligonucleotides corresponds to a mapped or known row location within the plurality of arrays. The first oligonucleotide complex (e.g., output of anneal (108)) can be extended as illustrated extend step (112) in FIG. 1A so that BC₂ and S₂′ of the second oligonucleotide (110) are copied to extend the first oligonucleotide portion. After extension, the output of the extend step (112) can be washed and melted (114) to form a single stranded oligonucleotide including the first and second oligonucleotide. In various embodiments, a ligase can be used to extend the first oligonucleotide so that a portion of the second oligonucleotide is copied.

Now that the second oligonucleotide has been coupled to the surface, a third oligonucleotide can be coupled to the surface by the anneal step (116), as shown in FIG. 1A. Another liquid containing a third oligonucleotide can be disposed onto each array of the plurality of arrays where a portion of the uncoated region in between neighboring arrays is not covered by the liquid containing the third oligonucleotide. The third oligonucleotide (118) can be coupled to the second oligonucleotide to form a second surface oligonucleotide complex, as illustrated by the output of the anneal step (116). The third oligonucleotide can include a unique column barcode sequence that is the same oligonucleotide sequence within a column and a different sequence between each of the columns of the plurality of arrays. Each of the unique column barcode sequences of the second oligonucleotides corresponds to a mapped or known column location within the plurality of arrays.

In various embodiments, the second oligonucleotide further includes a random barcode sequence that is a unique sequence for at least 99% of the formed second surface oligonucleotide complexes. The random barcode sequence may be referred to as a UMI or unique molecular identifier.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6^{th} edition (Saunders, 2007).

“Barcode” means a molecular label or identifier. In some embodiments, a barcode is a molecule attached to an analyte or a segment of an analyte (for example, in the case of polynucleotide barcodes and analytes) which may be used to identify the analyte. In some embodiments, a barcode (referred to herein as a “spatial barcode”) may be attached to a surface to identify a location on the surface. In some embodiments, populations of identical spatial barcodes may be disposed within a particular area on a surface. The size and shape of such areas may vary widely. In some embodiments, areas with unique spatial barcodes have the same magnitude and are disposed in a regular pattern on a surface with a density of spatial barcodes per unit area. In some embodiments, densities of such barcodes may vary form 1 barcode per mm² to 1000 barcodes per mm², or from 1 barcode per mm² to 500 barcodes per mm², or from 1 barcode to 200 barcodes per mm². In some embodiments, there may be a one-to-one correspondence between different spatial barcodes and different areas on a surface; that is, each different area may have a different and unique barcode. In some embodiments, the identity of a spatial barcode is determinable, for example, by sequencing whenever a spatial barcode is a polynucleotide. In some embodiments, a spatial barcode is an oligonucleotide. In some embodiments, an oligonucleotide spatial barcode comprises a random sequence oligonucleotide. A random sequence oligonucleotide is typically synthesized by a “split and mix” synthesis techniques, for example, as described in the following references that are incorporated herein by reference: Church, U.S. Pat. No. 4,942,124; Godron et al, International patent publication WO2020/120442; Seelig et al, U.S. patent publication 2016/0138086; and the like. Sometimes a random oligonucleotide is represented as “NNN . . . N.” In some embodiments, the term “barcode” includes composite barcodes; that is, an oligonucleotide segment that comprises sub-segments that identify different objects. For example, a first segment of a composite barcode may identify a particular area of a surface and a second segment of a composite barcode may identify a particular molecule (a so-called “unique molecular identifier” or UMI).

“Microfluidics” device or “nanofluidics” device, used interchangeably herein, each means an integrated system for capturing, moving, mixing, dispensing or analyzing small volumes of fluid, including samples (which, in turn, may contain or comprise cellular or molecular analytes of interest), reagents, dilutants, buffers, or the like. Generally, reference to “microfluidics” and “nanofluidics” denotes different scales in the size of devices and volumes of fluids handled. In some embodiments, features of a microfluidic device have cross-sectional dimensions of less than a few hundred square micrometers and have passages, or channels, with capillary dimensions, e.g. having cross-sectional dimensions of from about 1-2 mm to about 0.1 μm. In some embodiments, microfluidics devices have volume capacities in the range of from 100 μL to a few nL, e.g. 10-100 nL or in the range of from 100 μL to 1 μL. Dimensions of corresponding features, or structures, in nanofluidics devices are typically from 1 to 3 orders of magnitude less than those for microfluidics devices. One skilled in the art would know from the circumstances of a particular application which dimensionality would be pertinent. In some embodiments, microfluidic or nanofluidic devices have one or more chambers, ports, and channels that are interconnected and in fluid communication and that are designed for carrying out one or more reactions or processes, either alone or in cooperation with an appliance or instrument that provides support functions, such as sample introduction, fluid and/or reagent driving means, such as positive or negative pressure, acoustical energy, or the like, temperature control, detection systems, data collection and/or integration systems, and the like. In some embodiments, microfluidics and nanofluidics devices may further include valves, pumps, filters and specialized functional coatings on interior walls, e.g. to prevent adsorption of sample components or reactants, facilitate reagent movement by electroosmosis, or the like. Such devices may be fabricated as an integrated device in a solid substrate, which may be glass, plastic, or other solid polymeric materials, and may have a planar format for ease of detecting and monitoring sample and reagent movement, especially via optical or electrochemical methods. In some embodiments, such devices are disposable after a single use. In some embodiments, microfluidic and nanofluidic devices include devices that form and control the movement, mixing, dispensing and analysis of droplets, such as, aqueous droplets immersed in an immiscible fluid, such as a light oil. The fabrication and operation of microfluidics and nanofluidics devices are well-known in the art as exemplified by the following references that are incorporated by reference: Ramsey, U.S. Pat. Nos. 6,001,229; 5,858,195; 6,010,607; and 6,033,546; Soane et al, U.S. Pat. Nos. 5,126,022 and 6,054,034; Nelson et al, U.S. Pat. No. 6,613,525; Maher et al, U.S. Pat. No. 6,399,952; Ricco et al, International patent publication WO 02/24322; Bjornson et al, International patent publication WO 99/19717; Wilding et al, U.S. Pat. Nos. 5,587,128; 5,498,392; Sia et al, Electrophoresis, 24: 3563-3576 (2003); Unger et al, Science, 288: 113-116 (2000); Enzelberger et al, U.S. Pat. No. 6,960,437; Cao, “Nanostructures & Nanomaterials: Synthesis, Properties & Applications,” (Imperial College Press, London, 2004); Haeberle et al, LabChip, 7: 1094-1110 (2007); Ren et al, Acc. Chem. Res., 46(11): 2396-2406 (2013); Cheng et al, Biochip Technology (CRC Press, 2001); and the like.

What is claimed is:

1. A method of making a barcoded surface, the method comprising:
synthesizing a plurality of arrays, wherein:
(i) each array comprises a plurality of discrete reaction sites,
(ii) each discrete reaction site comprises a first oligonucleotide coupled to a surface of a solid support at a 5′ end of the first oligonucleotide,
(iii) the surface comprises an uncoated region in between neighboring arrays, the uncoated region comprising an area where no first oligonucleotides are coupled to the surface,
(iv) the first oligonucleotide includes a first unique array barcode sequence for each discrete reaction site location within the array,
(v) each of the first unique array barcode sequences of the first oligonucleotides corresponds to a mapped discrete reaction site location within the array; and
disposing a liquid containing a second oligonucleotide onto each array of the plurality of arrays where a portion of the uncoated region in between the neighboring arrays is not covered by the liquid containing the second oligonucleotide, wherein:
(vi) the second oligonucleotide couples to the first oligonucleotide to form a first surface oligonucleotide complex,
(vii) the second oligonucleotide includes a second unique barcode sequence, the second unique barcode sequence including a same sequence within a first sub-group of the plurality of arrays and a different sequence between each of the first sub-groups of the plurality of arrays,
(viii) each of the second unique barcode sequences of the second oligonucleotides corresponds to a mapped first sub-group location within the plurality of arrays.

2. The method of claim 1, further comprising:
disposing another liquid containing a third oligonucleotide onto each array of the plurality of arrays, wherein a portion of the uncoated region in between the neighboring arrays is not covered by the another liquid containing the third oligonucleotide, wherein:
(ix) the third oligonucleotide couples to the second oligonucleotide to form a second surface oligonucleotide complex,
(x) the third oligonucleotide includes a third unique barcode sequence, the third unique barcode sequence including a same sequence within a second sub-group of the plurality of arrays and a different sequence between each of the second sub-groups,
(xi) each of the third unique barcode sequences of the third oligonucleotides corresponds to a mapped second sub-group location within the plurality of arrays.

3. The method of claim 1, wherein the second oligonucleotide further includes a random barcode sequence that is a unique sequence for at least 99% of the formed second surface oligonucleotide complexes.

4. The method of claim 1, wherein the disposing the liquid containing the second oligonucleotide onto each array of the plurality of arrays comprises:
disposing a puddle of the liquid onto each array, wherein each puddle does not touch neighboring puddles.

5. The method of claim 4, wherein the puddle is formed by disposing the liquid as a plurality of dispensed droplets that merge together.

6. The method of claim 5, wherein the plurality of dispensed droplets have
a pitch along the x axis ranging from about 1 micron to about 40 microns, and
a pitch along the y axis ranging from about 1 micron to about 40 microns.

7. The method of claim 5, wherein each of the droplets of the plurality of dispensed droplets have (i) a diameter ranging from about 80 microns to about 140 microns or (ii) a volume ranging from about 200 picoliters to about 350 picoliters.

8. The method of claim 1, wherein the disposing the liquid containing the second oligonucleotide onto each array of the plurality of arrays comprises:
disposing the liquid as a plurality of droplets, wherein each droplet at least partly covers each of the discrete reaction sites.

9. The method of claim 1, wherein the synthesizing the plurality of arrays comprises coupling an amine moiety of the first oligonucleotide with the surface via a coupling layer, wherein an amine reactive moiety of the coupling layer binds to the amine moiety.

10. The method of claim 1, further comprising after coupling the second oligonucleotide to the first oligonucleotide to form the first surface oligonucleotide complex, adding a ligase to extend the first oligonucleotide so that a portion of the second oligonucleotide is copied.

11. The method of claim 1, wherein the plurality of arrays is arranged in rows and columns, and wherein the rows and the columns of the plurality of arrays are arranged in an orthogonal relationship.

12. The method of claim 1, wherein each of the discrete reaction sites of the array are formed from a single disposed droplet containing the first oligonucleotides.

13. The method of claim 1, wherein each of the arrays of the plurality have a same patterned location of the first unique array barcode sequences of the first oligonucleotide for each of the discrete reaction sites of the array.

14. The method of claim 1, wherein each of the discrete reaction sites of the array has a pitch between the reaction sites in the range of from 50-500 microns, and wherein the discrete reaction sites each have a diameter in the range of from 30-300 microns.

15. The method of claim 1, wherein each of the discrete reaction sites of the array has a density of reaction sites in the range of from 50 to 200 reaction sites per mm².

16. The method of claim 1, wherein the first sub-group is an array category selected from the group consisting of a row, a column, a diagonal, an even numbered row, an odd numbered row, an even numbered column, an odd numbered column, a first checkerboard pattern, a second checkerboard pattern, and combinations thereof.

17. The method of claim 2, wherein the second sub-group is an array category selected from the group consisting of a row, a column, a diagonal, an even numbered row, an odd numbered row, an even numbered column, an odd numbered column, a first checkerboard pattern, a second checkerboard pattern, and combinations thereof, wherein the array category of the first sub-group and the second sub-group are different.

18. The method of claim 1, wherein the first sub-group comprises the row, wherein the second unique barcode sequence includes a same sequence within the row of the plurality of arrays and a different sequence between each of the rows of the plurality of arrays.

19. The method of claim 2, wherein the second sub-group comprises the column, wherein the third unique barcode sequence includes a same sequence within the column of the plurality of arrays and a different sequence between each of the columns of the plurality of arrays.

20. A flow cell configured to capture cells from a biological sample in a hydrogel cage comprising:
(a) an inlet configured for receiving a liquid;
(b) an outlet configured outputting the liquid;
(c) a top layer;
(d) a spacer layer having a cut-out portion; and
(e) a bottom layer, wherein the top layer and the bottom layer are in an opposing relationship with the spacer layer in between the top layer and the bottom layer, wherein the top layer, the bottom layer, and the cut-out portion cooperate to form a channel, wherein the inlet and the outlet are in liquid communication with the channel allowing liquid flow through the channel, the bottom layer comprising a plurality of arrays, wherein
(i) each array comprises a plurality of discrete sites where a spatial barcode oligonucleotide is coupled to a surface on the bottom layer at each of the discrete sites, wherein the spatial barcode oligonucleotide comprises: a first oligonucleotide and a second oligonucleotide,
(ii) the surface includes an uncoated region in between neighboring arrays, the uncoated region comprising an area where no spatial barcode oligonucleotides are coupled to the surface,
(iii) the first oligonucleotide includes a first unique array barcode sequence for each discrete site location within the array,
(iv) the first unique array barcode sequence of the first oligonucleotide corresponds to a mapped discrete site location within the array;
(v) the second oligonucleotide includes a second unique barcode sequence, the second unique barcode sequence including a same sequence within a first sub-group of the plurality of arrays and a different sequence between each of the first sub-groups of the plurality of arrays, and
(vi) the second unique barcode sequence of the second oligonucleotide corresponds to a mapped first sub-group location within the plurality of arrays.

21. The flow cell of claim 20, wherein the second oligonucleotide further includes a random barcode sequence that is a unique sequence for at least 99% of the spatial barcode oligonucleotides.

22. The flow cell of claim 20, wherein the first sub-group is an array category selected from the group consisting of a row, a column, a diagonal, an even numbered row, an odd numbered row, an even numbered column, an odd numbered column, a first checkerboard pattern, a second checkerboard pattern, and combinations thereof.

23. The flow cell of claim 22, wherein the first sub-group comprises the row, wherein the second unique barcode sequence includes a same sequence within the row of the plurality of arrays and a different sequence between each of the rows of the plurality of arrays.

24. The flow cell of claim 20, further comprising: a third oligonucleotide coupled to the second oligonucleotide to form a second surface oligonucleotide complex, wherein the third oligonucleotide includes a third unique barcode sequence, wherein the third unique barcode sequence includes a same sequence within a second sub-group of the plurality of arrays and a different sequence between each of the second sub-groups, and wherein each of the third unique barcode sequences of the third oligonucleotides corresponds to a mapped second sub-group location within the plurality of arrays.

25. The flow cell of claim 24, wherein the second sub-group is an array category selected from the group consisting of a row, a column, a diagonal, an even numbered row, an odd numbered row, an even numbered column, an odd numbered column, a first checkerboard pattern, a second checkerboard pattern, and combinations thereof, wherein the array category of the first sub-group and the second sub-group are different.

26. The flow cell of claim 25, wherein the second sub-group comprises the column, wherein the third unique barcode sequence includes a same sequence within the column of the plurality of arrays and a different sequence between each of the columns of the plurality of arrays.
